(19) United States
(12) Patent Application Publication
(10) Pub. No.: US 2007/0010884 A1
(43) Pub. Date: Jan. 11, 2007

(54) PROSTHESES

(75) Inventor: Michael Antony Tuke, Guildford, Surrey (GB)

Correspondence Address: SENNIGER POWERS, ONE METROPOLITAN SQUARE, 16TH FLOOR, ST LOUIS, MO, 63102, US

(73) Assignee: FINSBURY (DEVELOPMENT) LIMITED, Leatherhead (GB)

(21) Appl. No.: 11/428,036

(22) Filed: Jun. 30, 2006

(30) Foreign Application Priority Data
Jul. 4, 2005 (GB) 0513686.6

(51) Int. Cl. A61F 2/08; A61B 17/17

(52) U.S. Cl. 623/13.14; 606/96; 623/13.12

(57) ABSTRACT

A replacement ligament prosthesis including an elongate replacement ligament having a femoral end and a tibial end. The prosthesis includes a bar, which in use will extend between the medial and lateral condyles of a femur and across the intercondylar notch, and to which the femoral end of the elongate replacement ligament will be fixed. The prosthesis also includes a fixing means, which in use will fasten the tibial end of the elongate replacement ligament to the tibia.

### BACKGROUND OF THE INVENTION

The present invention relates to a replacement anterior and/or posterior cruciate ligament in the knee. In addition, the present invention relates to a replacement collateral ligament for the knee. For ease of reference the replacement anterior and/or posterior cruciate ligament and the replacement collateral ligament will hereinafter be collectively referred to simply as the “replacement ligament”. More particularly, the present invention relates to a replacement cruciate ligament which is suitable for use with a prosthetic knee. In alternative arrangements, the present invention also relates to a guide to assist in the placement of a replacement of a cruciate ligament, a kit of components for use in the implantation of a replacement cruciate ligament and a method of inserting the replacement cruciate ligament.

The human knee is a complex joint which is stabilised by several ligaments. The four most important of which are the lateral and medial collateral ligaments and the anterior and posterior cruciate ligaments. FIGS. 1 and 2 illustrate the position of the lateral collateral ligament 1, the medial collateral ligament 2 and the posterior and anterior cruciate ligaments 3. These ligaments each extend from the femur 4 to the tibia 5. The normal human knee relies heavily on the posterior and anterior cruciate ligaments 3 for anterior-posterior stability. This stability means that there is little or no freedom of motion other than hinge movement of the knee during flexion and extension. Whilst there is some rotation, there will be little or no anterior-posterior laxity. The position of the femur in extension (position A) and in flexion (position B) is illustrated in FIG. 2. The location of the posterior and anterior cruciate ligaments in these positions is identified by dotted lines marked P1 and P2 respectively.

As the posterior and anterior cruciate ligaments are approximately centrally located in the medial and lateral direction in the knee, they allow controlled movement of the knee between the extension and flexion positions. It is also believed that the posterior and anterior cruciate ligaments work in conjunction with the medial collateral ligament in the overall anterior/posterior stability of the knee.

The posterior and/or anterior cruciate ligaments may become damaged through trauma, such as a sporting injury, or accident. The damage may be only a minor tear and may therefore in time heal. However, even if the tear heals successfully, the ligament may be too lax for correct functioning. Chronic cruciate ligament laxity or rupture may cause significant patella-femoral, and other knee function problems, due to instability of the femur on the tibia. More severe injuries can cause damage that will not heal to provide natural function and stability. Although ligament reconstructive techniques are performed relatively successfully in the unreplaced knee, they generally are difficult and may leave the patient in a worsening position than if there had been no treatment.

Where a patient has to undergo replacement knee surgery, the surgeon may retain and utilise the posterior cruciate ligament. However, it is rare for the anterior cruciate ligament to be retained for a total knee replacement. Further the retained posterior ligament is rarely of the the appropriate length to function correctly. It is therefore common for surgeons to remove all remnants of the anterior and posterior cruciate ligaments rather than retain them. One or both of the ligaments may also be removed in situations where the surgeon believes that retention will conflict with the motion of the replacement knee joint.

Prostheses for uni-compartmental or total knee replacement may have some laxity in flexion, i.e. anterior/posterior mechanical instability. This is usually due to the implant having no intrinsic anterior/posterior stability. Additionally or alternatively poor placement of the implants will result in laxity in either, or both of, extension and flexion. In an attempt to address the problems associated with these knee prostheses and surgical flaws, alternative prostheses have been developed which are said to provide “posterior stabilisation”. These knee prostheses generally include an upstanding polyethylene post located on the upper surface of the tibial component on the knee prosthesis and a central cam located between the condyles on the femoral component of the prosthesis.

FIGS. 3 to 5 illustrate one example of a prosthesis of this type. The position of the post 5 on the tibial component 6 and its relationship with the cam 7 on the femoral component 8 are illustrated schematically in side view in FIG. 3 and in front view in FIG. 4. As the leg is moved, and the knee flexed, the femoral component 8 rotates until the cam 7 engages the post 5.

The interaction of the cam 7 with the post to prevent further rotation as illustrated in FIG. 5. Once the cam 7 has engaged the post 5, any anterior movement by the femoral component on the tibial component is prevented. In some cases, the engagement with the post 5 forces a posterior motion of the femur on the tibia.

These prostheses are popular as they do provide some stability by enhancing the degree of flexion in lax knees by ensuring that the femur is placed relatively posteriorly on the tibia as the degree of flexion increases. However, the stability is only provided for a limited range of motion. In particular, the level of stability required for walking may not be provided. This is because the post and cam are not articulating together during the walking range of motion.

A further disadvantage of these prostheses is that the forces that the post and cam mechanism exert are not easy to predict or measure and are therefore not designed to resist for long implantation periods. This can lead to the parts wearing badly or even fracturing. This wear and stress can lead to the fixation between the implant and the bone being too high or changing, which in turn may lead to extra loosening loads being experienced. This in turn will lean to relatively early failure of the joint.

A still further disadvantage of posterior stabilised knee prostheses is that in order to insert the prostheses, the knee must be relatively lax. Thus the insertion of the knee prostheses may actually ensure or exacerbate the problems detailed above. Mobile tibia meniscus designs for knee replacements are now evolving which include such cam and post arrangements. However, these add a further engineering complexity that predisposes their failure.

Various attempts have been made to provide replacement ligaments for the normal or near-normal un-replaced knee. In one arrangement these replacement ligaments are natural ligaments harvested from another site in the patient. Whilst such ligaments may be useful, the harvesting step increases the cost of the operation and the suffering to the patient.

As an alternative to replacement natural ligaments for the normal knee artificial ligaments may be provided formed from plastics materials such as polyester. However, known artificial ligaments are generally unable to deal with the stresses and fatigue caused in the ligament at the femoral insertion point due to sustained repeated flexural motion.

Whilst replacement ligaments have offered some success with natural knee joints, artificial replacement ligaments have not generally been provided which can successfully be used with prosthetic knees.

The knee is expected to have a range of motion of up to about 150 degrees from full extension to full flexion. Any ligament must therefore be able to withstand this repeated angular displacement whilst under load. Most of the angular motion is concentrated at the insertion point of the ligament into the femur and hence the material properties are highly tested, man made fibres being vulnerable to flexural stress and fatigue failure. The placement of this insertion point is very critical for the knee to function correctly and to optimise the function of the ligament replaced. This placement is related to the articular surfaces of the femur and tibia which interact to provide instant centres of motion on the femur through which the ligament should be placed.

It is therefore desirable to provide replacement cruciate ligaments which can improve knee stability and reduce unwanted anterior-posterior movement of the femur when the knee is flexed. It is particularly desirable to provide replacement cruciate ligaments which may be successfully used with partial (uni-compartmental) or total prosthetic knee joints. It is further desirable to be able to avoid flexural failure and to place the femoral end of the ligament very accurately with respect to the articular surfaces of the knee throughout its motion. It is further desirable to provide replacement collateral ligaments.

The problems associated with prior art arrangements may be overcome by providing a replacement ligament and means for correctly connecting the ligament to the femur and tibia.

### SUMMARY OF THE INVENTION

Thus, according to the present invention there is provided a replacement ligament prosthesis for the knee comprising:

an elongate replacement ligament having a femoral end and a tibial end;

an elongate replacement ligament having a femoral end and a tibial end;

a bar, which in use will extend between the medial and lateral condyles of a femur and across the intercondylar notch, and to which the femoral end of the elongate replacement ligament will be fixed; and

fixing means, which in use will fasten the tibial end of the elongate replacement ligament to the tibia.

The ligament prosthesis of the present invention may be used in connection with any non replaced knee with a deficient ligament or any type of surface replacing knee implant prostheses including partial and total knee replacement prostheses. Thus in the following discussion, reference to the femur or tibia and parts thereof may be natural components or prosthetic replacements therefor.

The elongate replacement ligament may be of any suitable configuration. In one arrangement, the replacement ligament may be formed from a plurality of fibres that may be combined to form the ligament in any suitable manner. In one arrangement, the fibres may be twisted together to form a rope-like structure.

The replacement ligament may be formed from any suitable material. In general, the material from which the replacement ligament is formed will have high strength in tension. Suitable materials include natural fibres such as muscle fascia, or synthetic fibres such as polyester, Dacron, nylon, carbon, Kevlar and the like. These materials may be used alone or in combination.

The replacement ligament may be of any suitable size. It may have a cross-sectional diameter of from about 2 to about 10 mm. In one arrangement, it may have a cross-sectional diameter of from about 4 to about 8 mm and preferably will be about 6 mm.

The length of the replacement ligament will be selected to be appropriate for the patient being treated. In general it will be from about 50 to about 250 mm in length. It is preferred that in use, the replacement ligament will pass through a bore in the tibia from the posterior surface to the anterior surface and will then be fixed at the anterior surface. It is therefore desirable that the replacement ligament will be of sufficient length to achieve this. However, it will generally be provided of longer length and then when fitted will be cut to the correct length.

As detailed above, in use, the replacement ligament may be passed through a bore in the tibia. To assist in this feeding, the tibial end of the elongate replacement ligament may be provided with a leader line. The leader line may be made of any suitable material. In an alternative arrangement, the leader line may be separate and in use passed through a folded portion of the replacement ligament to assist in feeding the ligament through a bore in the tibia. Suitable materials include natural fibres such as muscle fascia, or synthetic fibres such as polyester, Dacron, nylon, carbon, Kevlar and the like. Mixtures of these fibres may also be used. The cross-sectional diameter of the leader will generally be less than that of the elongate replacement ligament. Whilst any suitable diameter may be used, these will generally be in the range of from about 0.5 mm to about 3 mm.

In use, the replacement ligament may be attached to the bar by any suitable means. In one arrangement, the femoral end of the replacement ligament may be provided with a loop which in use will be passed over the bar. The loop may be integral with the replacement ligament or may be provided as a separate component which is connected to the ligament. In one arrangement, where the replacement ligament is formed from a stranded material such as when it is a rope-like material, the loop may be formed by splicing the ligament. Where a loop is present, a central bush may be present.

Where a loop is present, it will be of any suitable size. In one arrangement, it may be from about 2 to about 10 mm in diameter. More preferably, it may have a diameter of from about 4 to about 8 mm, and most preferably, a diameter of about 6 mm.

One alternative means for attaching the replacement ligament to the bar is simply tying the femoral end of the replacement ligament to the bar.

In a further alternative arrangement the replacement ligament may be attached to the bar by means of a locking means provided on the replacement ligament which can engage the bar. Examples of suitable locking means include eyelets, clamps and the like.

In a still further alternative arrangement, the means for connecting the replacement ligament may be provided on the bar. For example, the bar may include a notch into which the ligament may be engaged. In another arrangement an aperture may be provided in the bar through which the ligament can be passed. In this latter arrangement the replacement ligament may be provided with a pivotable pin which can be threaded through the aperture and then pivoted to a position which will prevent the ligament from being pulled back through the aperture.

As in use the bar will be located at the end of the femur so that it extends between the medial and lateral condyles of the femur, it will be understood that it is an elongate component. It may be of any configuration but in one arrangement may have a circular cross-section. Alternative arrangements the shape of the bar in cross-section may differ along its length.

The bar may be formed of any suitable material. Suitable materials include metals, ceramics and plastics. However, metals are generally preferred. One suitable metal is cobalt chrome.

In one arrangement, the bar may be provided with anti-movement means which will prevent one or more of rotation, medial-lateral movement, and anterior-posterior movement, when located in the intercondylar notch.

The anti-movement means may be formed by any suitable arrangement. Where the movement to be prevented is rotation, the bar may be configured such that it has a cross-section which will prevent rotation once the bar is in situ. Thus, the bar may be of non-circular cross-section. In one arrangement, the bar may be of generally circular cross-section in a central region and of non-circular cross-section at the ends.

In one arrangement, the anti-movement means may be provided by means of one or more fins extending from the bar which in use engage with the bone or prosthesis.

The bar may be held in place by any suitable means. In one arrangement, brackets may be located on the inner wall of the condyles into which the ends of the bar may be located. In a preferred arrangement, one or both ends of the bar will be located in a bore in the respective condyles. The bores communicate with the intercondylar notch. One or both bores may extend through the condyle. In one arrangement, the bore may extend through one condyle so that the bar may be inserted through the bore, across the intercondylar notch and into a corresponding bore in the other condyle. The bore in the other condyle will preferably not extend completely through the condyle. In this preferred arrangement, the bar may be a screw. In one arrangement it will be a self-tapping screw such that it is not necessary to pre-form the bore in the other condyle.

Where the bar is formed from a screw, the threads of the screw may serve as the anti-movement fins.

The tibial end of the elongate replacement ligament may be fixed to the tibia by any suitable fixing means. In one arrangement, the tibial end may be connected to the tibia by a staple, nail or screw. Where the fixing means is a screw, it is preferably a non-sharp edged screw.

It will be understood that when in position, the portion of the bar lying in the intercondylar notch will preferably be fully exposed. Generally, the portion of the bar in the intercondylar notch will be free from anti-movement means. In a preferred arrangement this portion of the bar will be cylindrical and polished such as that in arrangements when a loop is present on the elongate replacement ligament, it can seat around the bar.

It will be understood that for the correct functioning of the replacement ligament, the bar is preferably located to lie on the centre of rotation of the knee femur as determined by the articular surfaces of the femur whether a natural or replacement knee femur. Where the bar is to be placed in a bore in the condyle, it may be desirable to utilise a jig to guide the axial direction of the drill which will be used to produce the bore into which the bar will be placed. However, in some circumstances it may be desirable for the surgeon to select a different position for the bar where that will provide improved functioning. For example, a planned eccentricity for the posterior cruciate ligament can induce so called “roll back” of the femur on the tibia and a planned eccentricity for the anterior cruciate ligament may induce “roll forwards”.

A remote navigation system may be used prior to surgery to track the motion of the knee and determine the appropriate axis on which the bar should be placed.

Where a jig is used, it will preferably allow the surgeon to provide the bore for the bar at a selected but defined position.

Where more than one ligament is to be replaced, they may be attached to the same bar or in one arrangement located at different bars located on different axis through the knee joint.

According to a second aspect of the present invention there is provided a jig for use in locating a selected condylar axis comprising:

two spaced rods extending radially from a guide; and

a finger extending from the distal end of each rod and being located substantially at right angles thereto such that the fingers are parallel and are adapted in use to be located against the femoral condyles of a flexed femur,

the rods being of the same length which is selected such that when the fingers are located against the femoral condyles, the guide is aligned approximately on the epicondylar axis or flexion axis of the knee.

Without wishing to be bound by any particular theory, there has been some discussion that the epicondyler axis and the flexion axis of the knee are not collinear. Discussions as to, if they are different, which is the correct one relevant to knee function are ongoing.

In use the jig will generally be located on the femur in maximum flexion and the fingers will sit on the femoral condyles to reference the distal and about 60° to about 90° degrees into flexion surfaces of the femur. By this means the convergence of the tangents to these two angular positions locates the centre point on the medial condyle. In a natural knee this point may be referred to as the medial epicondyle. Where a replacement knee is present, the point may be referred to as a new artificially created epicondyle based upon the new knee implant. The guide may also be used to pinpoint the lateral epicondyle.

The guide may be of any suitable shape but will generally be of substantially circular configuration. In one arrangement it may be formed as only part of a circle. In use the surgeon may use the guide to simply mark the position of the axis. However, in a preferred arrangement, the guide is a drill guide and therefore is configured to ensure that the drill is correctly lined up on the axis.

The rods may be of any suitable size. In one arrangement, the rods may be between about 15 mm and about 30 mm. The fingers may also be of any suitable size. A suitable size is generally about 60 mm and about 120 mm. Particularly suitable combinations are: rods of about 20 mm in length with fingers of about 60 mm in length; rods of about 24 mm in length with fingers of about 70 mm in length; or rods of about 26 mm in length with fingers of about 80 mm in length. In one alternative arrangement, the rods and/or the fingers may be adjustable in length. For example, they may be telescopic.

The jig may be formed of any suitable material although it will preferably be formed from metal.

In use the jig will generally be stabilised. Stabilisation may be provided by means of pins. In one arrangement, it may be clamped to at least one of the condyles, for example to either side of the femoral condyles.

According to a third aspect of the present invention there is provided a kit of parts comprising the components of a replacement ligament prosthesis wherein the replacement ligament prosthesis is the prosthesis of the above first aspect of the present invention.

The kit may additionally include a partial or total replacement knee prosthesis.

The kit may additionally include a wire finder.

The kit may additionally include a jig. The jig may be the jig of the above second aspect of the present invention.

In order to introduce the replacement ligament of the first aspect of the present invention, the knee, whether natural or prosthetic will be flexed to expose the femoral condyles. The epicondylar axis or the flexion axis may then be located preferably utilising the jig of the above second aspect of the present invention. Where the bar is to be inserted in a bore which extends through one condyle, a drill may be used to create a bore along the epicondylar axis or the flexion axis. It is likely that the drill will pass through the medial condyle, through the inter-condylar notch and into the lateral condyle. It will be understood that the drill could be operated from the lateral condyle side however, this will not generally be preferred.

The femoral bore may or may not pass through the entirety of the lateral condyle. The diameter of the bore will be selected to correspond to the bar which is to be introduced. In one arrangement, the drill bit may have steps of different diameter along its length or be performed in stages using different drill bits so that different diameters are obtained for different sections of the bone. The bar will then be introduced into the bore. Where the bar is a screw, it will be screwed into position.

Where the ligament is to be connected to the bar by means of a loop in the ligament, an eyelet or a boss, the bar will be passed through the loop or other means as it is passed through the intercondylar notch.

A tibial bore is produced which extends from the anterior surface of the tibia to the posterior surface of the tibia for the posterior ligament and similarly as appropriate and normally exercised for an anterior ligament. If both the anterior and posterior ligaments are being replaced the bar may accommodate both ligaments or their respective boss/eyelets. In one alternative arrangement the two ligaments may be connected before they are connected with the bar. In one arrangement, they may both surround a single eyelet or boss. The position where the tibial bore emerges on the surface of the tibia will be the position at which the ligament would naturally be joined to the tibia. The tibial bore may be produced by any suitable means. It will be understood that the bore may be produced by drilling in either direction.

The ligament member is passed through the tibial bore. This may be done using a wire finder. Before the end of the ligament member is fixed to the tibia, the surgeon will generally make an assessment of the optimum tension of the ligament member. This may involve taking up the slack in the ligament member and testing the knee in tension and flexion to determine the optimal length of the ligament member.

The ligament member is then fixed to the tibia using the fixing means. Fixing may be achieved by inserting the fixing means into the drilled hole such that the fixing means grips the ligament member tightly against the inner wall of the tibial bore. However, any means of permanently securing the tibial end of the ligament member to the tibia may be employed in the present invention.

It will be understood that the above steps may take place in any suitable order. For example the bones in the femur and tibia may both be formed before the components of the replacement ligament prosthesis are used.

According to a fourth aspect of the present invention, there is provided a method of implanting the ligament prosthesis of the above first aspect which comprises:

i) locating an epicondylar axis;

ii) providing a femoral bore approximately on the epicondylar axis or flexion axis into the femur;

iii) making a tibial bore which extends from the anterior surface of the tibia to the posterior surface of the tibia;

iv) passing the ligament through the tibial bore;

v) locating the bar in the bore;

vi) coupling the bar and the elongate replacement ligament; and

vii) fixing the ligament to the tibia using fixing means.

The components of the ligament prosthesis are preferably those of the above first aspect.

### BRIEF DESCRIPTION OF THE DRAWINGS

The present invention will now be described, by way of example, with reference to the following examples in which:

FIG. 1 is an anterior-posterior view of the knee joint;

FIG. 2 is a medial-lateral view of the knee joint;

FIG. 3 is a schematic medial-lateral view of a knee prosthesis of the prior art in tension;

FIG. 5 is a anterior-posterior view of a knee prosthesis of the prior art in flexion;

FIG. 6 is the schematic illustration of the jig of the second aspect of the present invention;

FIG. 7 is a medial-lateral view of a knee in flexion with the jig in position;

FIG. 8 is an anterior-posterior view of FIG. 7;

FIG. 9 is a medial-lateral view of a knee in flexion including a replacement posterior cruciate ligament of the present invention; and

FIG. 10 is an anterior-posterior view of FIG. 9.

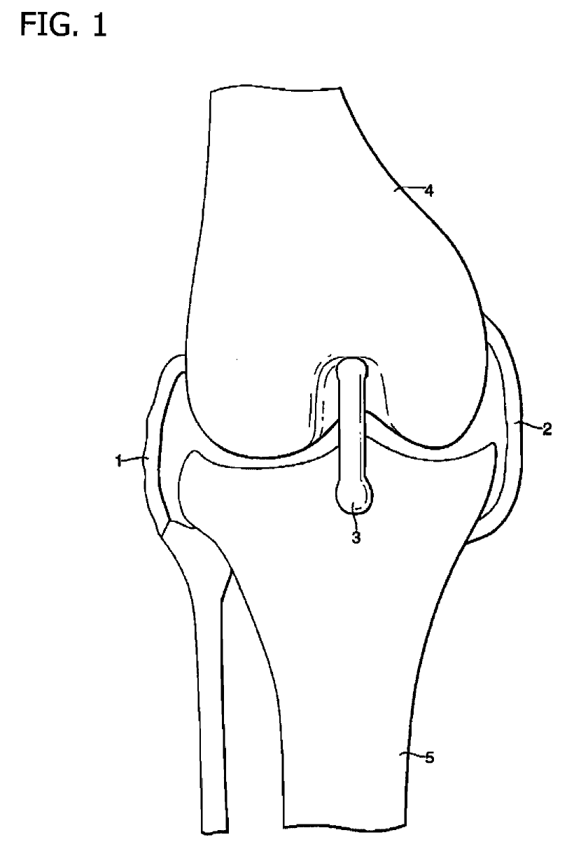

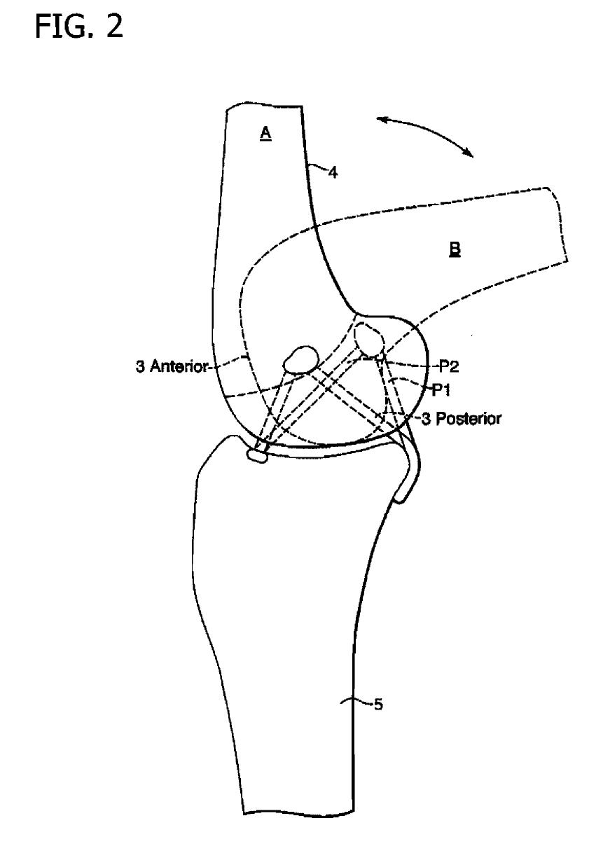

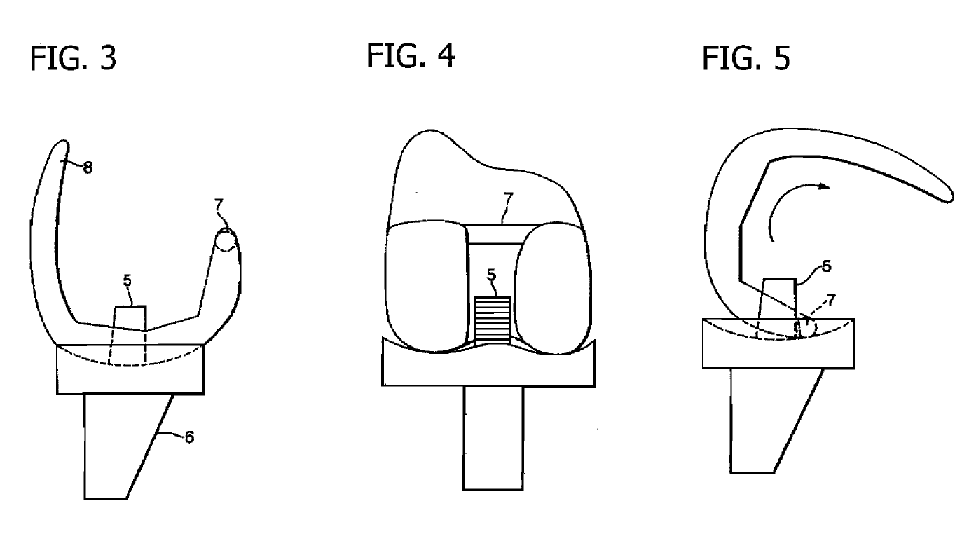

### DETAILED DESCRIPTION OF THE PREFERRED EMBODIMENTS

The present invention will now be described with reference to a knee prosthesis. However, it will be understood that it may be utilised with a normal knee.

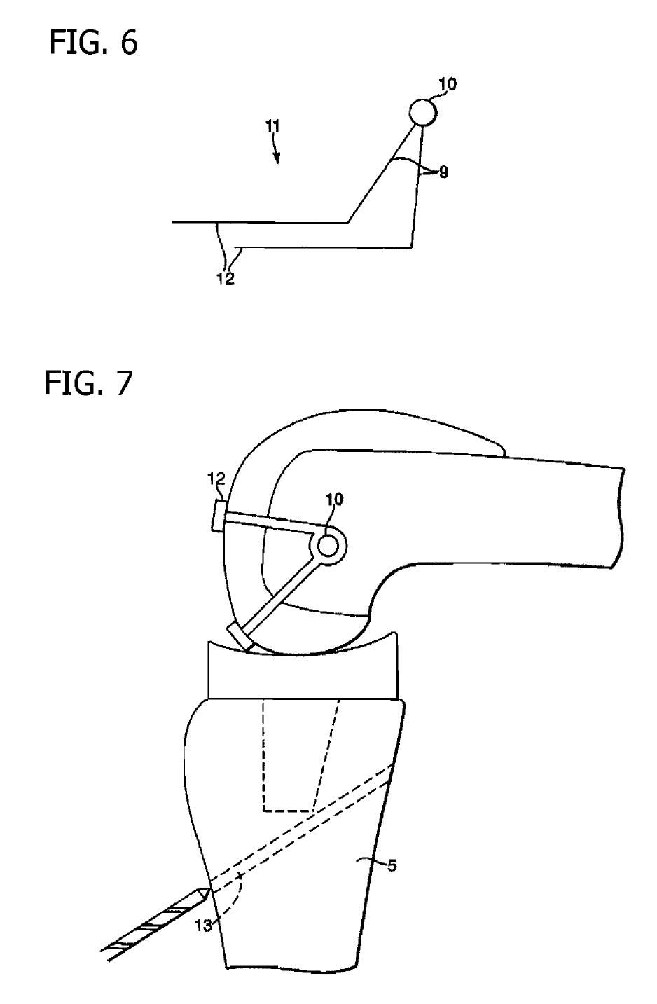

As illustrated in FIG. 6 a jig 11 comprises rods 9 connected to a guide 10. These are angled such they represent the arms of a segment of a circle. The arms are in the same plane and are of the same length. Fingers 12 extend from the end of the arms.

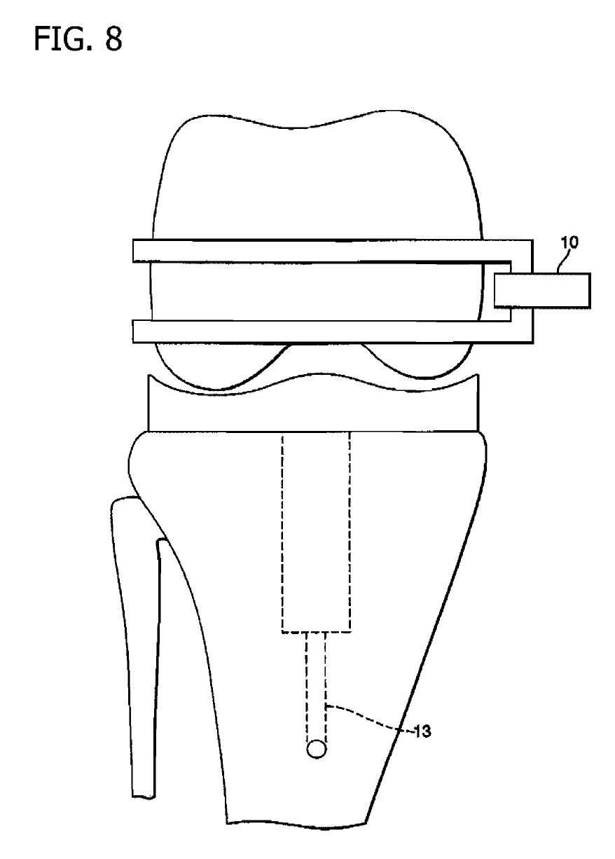

The jig 11 is then placed against the exposed knee which is positioned in flexion as illustrated in FIGS. 7 and 8. The fingers 12 are located against the face of the condyles with an upper finger being placed at the end of the femur 3. The aperture 10 lies on the epicondylar axis. As illustrated in FIG. 8, the aperture may be a drill guide.

Once the drill guide 12 is correctly positioned, it may be stabilised using pins or may be clamped on either side of the inter-condylar notch or the femur. The femoral bore will then be made and the jig removed.

A tibial bore 13 is provided to extend from the anterior surface of the tibia 5 to the posterior surface thereof.

A femoral loop end of the replacement ligament 21 is passed through the bore 22 in the tibia using a wire finder (not shown).

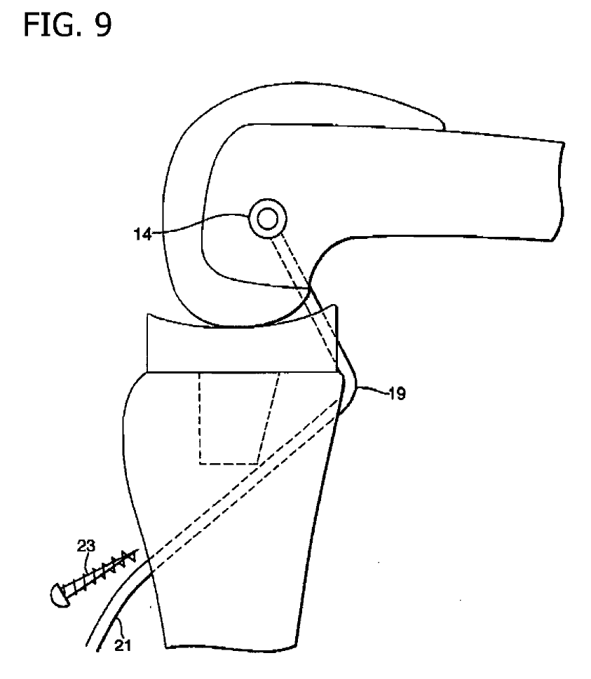

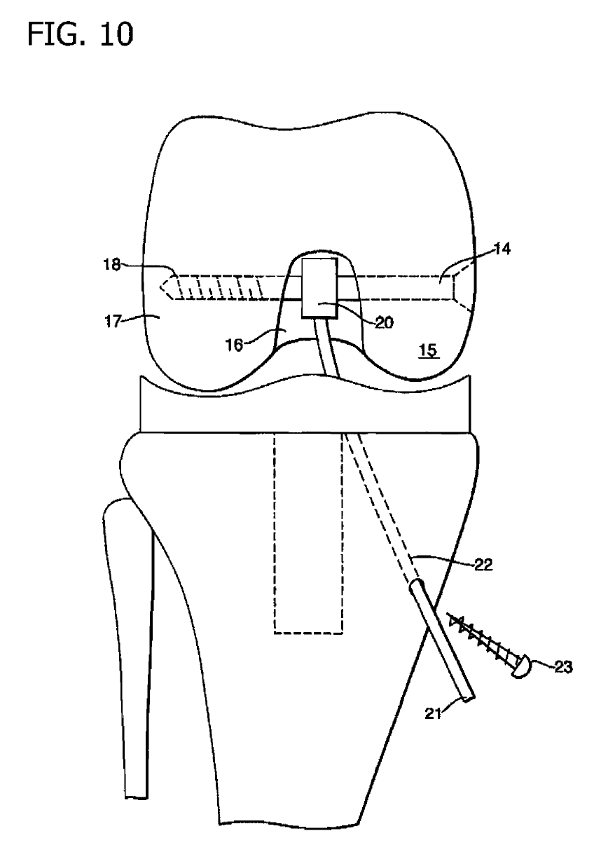

As illustrated in FIGS. 9 and 10, the bar 14 is inserted into the bore. The bar 14 is passed through the entirety of the medial condyle 15. The replacement ligament 19 has a loop 20 at the femoral end thereof through which the bar is passed and the bar then further inserted across the inter-condylar notch 16 and into the lateral condyle 17. The bar 14 includes fins provided by the screw-threads 18 on the bar to secure it in the bone of the femur.

The replacement ligament 19 has a loop 20 at the femoral end thereof through which the bar is passed.

Once the surgeon has made an assessment of the optimum tension for the replacement ligament, the ligament is fastened to the tibia using a screw 23. In one arrangement, the screw 23 is passed up the tibial bore 22 to fix ligament 21 in place. Any excess ligament 21 will be cut and removed.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above constructions, products, and methods without departing from the scope of the invention, it is intended that all matter contained in the above description and shown in the accompanying drawings shall be interpreted as illustrative and not in a limiting sense.

What we claim is:

1. A replacement ligament prosthesis comprising:
an elongate replacement ligament having a femoral end and a tibial end;
a bar, which in use will extend between the medial and lateral condyles of a femur and across the intercondylar notch, and to which the femoral end of the elongate replacement ligament will be fixed; and
fixing means, which in use will fasten the tibial end of the elongate replacement ligament to the tibia.

2. A replacement ligament prosthesis according to claim 1 for use with the natural knee.

3. A replacement ligament prosthesis according to claim 1 for use with knee prostheses.

4. A replacement ligament prosthesis according to claim 1 wherein the ligament is a posterior cruciate ligament.

5. A replacement ligament prosthesis according to claim 1 wherein the elongate replacement ligament is formed from a plurality of fibres.

6. A replacement ligament prosthesis according to claim 5 wherein the fibres may be twisted together to form a rope-like structure.

7. A replacement ligament prosthesis according to claim 1 wherein the replacement ligament has a cross-sectional diameter of from about 2 to about 10 mm.

8. A replacement ligament prosthesis according to claim 7 wherein the replacement ligament has a cross-sectional diameter of from about 4 to about 8 mm.

9. A replacement ligament prosthesis according to claim 7 wherein the replacement ligament has a cross-sectional diameter of about 6 mm.

10. A replacement ligament prosthesis according to claim 1 wherein the replacement ligament has a length of from about 50 to about 250 mm in length.

11. A replacement ligament prosthesis according to claim 1 wherein the elongate replacement ligament is provided with a leader line.

12. A replacement ligament prosthesis according to claim 1 wherein the femoral end of the replacement ligament is provided with a loop which in use will be passed over the bar.

13. A replacement ligament prosthesis according to claim 12 wherein the loop is integral with the replacement ligament.

14. A replacement ligament prosthesis according to claim 13 wherein the loop is formed by splicing the ligament.

15. A replacement ligament prosthesis according to claim 14 wherein a central bush is present.

16. A replacement ligament prosthesis according to claim 12 wherein the loop is from about 2 to about 10 mm in diameter.

17. A replacement ligament prosthesis according to claim 16 wherein the loop has a diameter of from about 4 to about 8 mm.

18. A replacement ligament prosthesis according to claim 17 wherein the loop has a diameter of about 6 mm.

19. A posterior cruciate ligament prosthesis according to claim 1 wherein the bar is provided with anti-movement means which prevents one or more of rotation, medial-lateral movement, and anterior-posterior movement, when located in the intercondylar notch.

20. A posterior cruciate ligament prosthesis according to claim 19 wherein the anti-movement means is provided by means of one or more fins extending from the bar which in use engage with the bone or prosthesis.

21. A posterior cruciate ligament prosthesis according to claim 20 wherein bar is formed from a screw and the threads of the screw serve as the anti-movement fins.

22. A posterior cruciate ligament prosthesis according to claim 1 wherein the fixing means is a staple, nail or screw.

23. A posterior cruciate ligament prosthesis according to claim 20 wherein the fixing means is a non-sharp edged screw.

24. A posterior cruciate ligament prosthesis according to claim 1 wherein the portion of the bar which will be located in the intercondylar notch is cylindrical and polished.

25. A jig for use in locating the epicondylar axis comprising:
two spaced rods extending radially from a guide; and
a finger extending from the distal end of each rod and being located substantially at right angles thereto such that the fingers are parallel and are adapted in use to be located against the femoral condyles of a flexed femur,
the rods being of the same length which is selected such that when the fingers are located against the femoral condyles, the guide being aligned approximately on the epicondylar axis or flexion axis of the knee.

26. A jig according to claim 25 wherein in use the fingers will sit on the femoral condyles to reference the distal and about 60° to about 90° degrees into flexion surfaces of the femur.

27. A jig according to claim 25 wherein the guide is a drill guide.

28. A kit of parts comprising the components of a replacement ligament prosthesis, wherein the replacement ligament prosthesis comprises:
an elongate replacement ligament having a femoral end and a tibial end;
a bar, which in use will extend between the medial and lateral condyles of a femur and across the intercondylar notch, and to which the femoral end of the elongate replacement ligament will be fixed; and
fixing means, which in use will fasten the tibial end of the elongate replacement ligament to the tibia.

29. A kit according to claim 28 wherein the kit additionally includes a partial or total replacement knee prosthesis.

30. A kit according to claim 28 wherein the kit additionally includes a wire finder.

31. A kit according to claim 28 wherein the kit additionally includes a jig for use in locating the epicondylar axis comprising:
two spaced rods extending radially from a guide; and
a finger extending from the distal end of each rod and being located substantially at right angles thereto such that the fingers are parallel and are adapted in use to be located against the femoral condyles of a flexed femur,
the rods being of the same length which is selected such that when the fingers are located against the femoral condyles, the guide being aligned approximately on the epicondylar axis or flexion axis of the knee.

32. A method of implanting a replacement ligament prosthesis comprising an elongate replacement ligament having a femoral end and a tibial end, a bar, which in use will extend between the medial and lateral condyles of a femur and across the intercondylar notch, and to which the femoral end of the elongate replacement ligament will be fixed, and a fixing means, which in use will fasten the tibial end of the elongate replacement ligament to the tibia, said method comprising:
i) locating an epicondylar axis;
ii) providing a femoral bore approximately on the epicondylar axis or flexion axis into the femur;
iii) making a tibial bore which extends from the anterior surface of the tibia to the posterior surface of the tibia;
iv) passing the ligament through the tibial bore;
v) locating the bar in the bore;
vi) coupling the bar and the elongate replacement ligament;
vii) fixing the ligament to the tibia using said fixing means.
